(19) United States
(12) Patent Application Publication
(10) Pub. No.: US 2010/0113426 A1
(43) Pub. Date: May 6, 2010

(54) Intranasal Benzodiazepine Compositions

(76) Inventor: Daniel P. Wermeling, Lexington, KY (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/612,352

(22) Filed: Nov. 4, 2009

Related U.S. Application Data
(63) Continuation of application No. 10/803,521, filed on Mar. 17, 2004, now abandoned.
Application No. 10/803,521 is a continuation-in-part of application No. 10/418,260, filed on Apr. 15, 2003, now abandoned.
Application No. 10/418,260 is a continuation of application No. 09/790,199, filed on Feb. 20, 2001, now Pat. No. 6,610,271.

(51) Int. Cl. A61K 31/5517; A61P 23/00

(52) U.S. Cl. 514/220

(57) ABSTRACT

A pharmaceutical composition for intranasal administration to a mammal. The pharmaceutical composition comprises an effective amount of a benzodiazepine or pharmaceutically acceptable salt thereof; and a nasal carrier. In some embodiments, the pharmaceutical composition when administered intranasally produces a rapid physiological response. Pharmaceutical compositions may also include at least one or more sweeteners, flavoring agents, or masking agents or combinations thereof.

This application is a continuation-in-part of U.S. application Ser. No. 10/418,260 filed Apr. 15, 2003, which is a continuation application of U.S. application Ser. No. 09/790,199 filed Feb. 20, 2001, now U.S. Pat. No. 6,610,271. The entire disclosure of these applications is herein incorporated by reference.

### BACKGROUND

Benzodiazepines have been used to prevent or treat a wide variety of clinical conditions based on their anxiolytic, hypnotic, anticonvulsant, and antispastic properties. Some benzodiazepines have also demonstrated efficacy for their antipanic, antidepressant, amnestic, and anesthetic effects.

Chlordiazepoxide and diazepam, the earliest benzodiazepines, have the classic 1,4-diazepine ring structure and also a 5-aryl substituent ring fused to a benzene ring. A number of modifications to the 1,4-diazepine structure led to compounds such as midazolam, which is a short-acting benzodiazepine that has an imidazo ring fused to the diazepine ring, and alprazolam and triazolam, which have a triazolo ring fused to the diazepine ring. There are other compounds that do not have the classic benzodiazepine structure, yet still have the anxiolytic or sedative effects associated with some of the benzodiazepines. These other compounds include for example, zopiclone, zolpidem, abecarnil, and bretazenil.

The therapeutic effects of benzodiazepines and other compounds, in part, result from enhancing the actions of the inhibitory neurotransmitter gamma-aminobutyric acid (GABA) at its receptor. Benzodiazepines work at the GABA receptor and cause GABA to produce a more rapid pulsatile opening of the chloride channel causing an influx of chloride into the cell.

Benzodiazepines have different onset and duration of action, making them useful in treating a variety of different clinical conditions. Benzodiazepines with short onset and duration of action may be useful when an immediate effect is needed (e.g., for outpatient surgical and diagnostic procedures), although longer duration of action may be desired (e.g., in treatment of sleep-maintenance disturbances or for seizure control). Some benzodiazepines have been used to treat anxiety, schizophrenia, phobias, sleep and depressive disorders. Used alone or in combination with neuroleptics, benzodiazepines have proved valuable for management of various psychiatric emergencies involving agitation or hostility. Intravenous diazepam is frequently a life saving drug in various convulsive emergencies, such as status epilepticus or tetanus spasms. Benzodiazepines frequently bring substantial relief of spasticity and involuntary movement disorders, such as, choreas, myoclonus, and some dyskinesias and dystonias associated with use of neuroleptic medications. Benzodiazepines are also effective in managing acute withdrawal from alcohol. When administered prior to surgical procedures, benzodiazepines reduce anxiety, provide sedation, facilitate anesthetic induction, and produce amnesia for the events surrounding induction. In the treatment of cancer, lorazepam and other benzodiazepines can help to control nausea and vomiting associated with chemotherapy.

Although benzodiazepines can be used to treat a wide variety of conditions, a patient's non-compliance or failure to take medication as prescribed, has been linked to inadequate treatment of many conditions. Some benzodiazepines are available by injections (e.g., intravenous (IV), intramuscular (IM) or subcutaneous injection). The intravenous route is normally regarded as one of the most in-convenient routes to administer medication. Intravenous administration may cause non-compliance, because not only do patients fear getting the injection, but unpleasant experiences such as pain, irritation and infection resulting at the injection site may also lead to non-compliance.

The intranasal route is currently receiving special interest for administering benzodiazepines. When medication is administered via the intranasal route, the medication is applied to the nasal mucosa where it is absorbed. The extensive network of blood capillaries under the nasal mucosa is particularly suited to provide rapid and effective systemic absorption of drugs. The intranasal route of administration should achieve similar dose to plasma concentration (bioavailability) and efficacy to that of the intravenous route.

Intranasal administration of medication provides numerous advantages over the intravenous route. The principal advantages of intranasal route are non-invasive delivery, rapid drug absorption, and convenience. The intravenous route, unlike the intranasal route, requires sterilization of hypodermic syringes and, in the institutional setting, leads to concerns among medical personnel about the risk of contracting disease if they are accidentally stuck by a contaminated needle. Strict requirements for the safe disposal of needles and syringes have also been imposed.

In contrast, intranasal administration requires little time on the part of the patient and attending medical personnel, and is far less burdensome on the institution than injectable routes. There is no significant risk of infection of the patient or medical personnel in the institutional setting when dealing with the intranasal delivery of medication.

A second important advantage of intranasal administration over intravenous is patient acceptance of the intranasal delivery route. In some cases, the injections cause burning edema, swelling, turgidity, hardness and soreness. In contrast, intranasal administration is perceived as non-invasive, is not accompanied by pain, has no after-effects and produces a prompt means of treating a wide variety of medical conditions. This is of particular advantage when the patient is a child. Many, if not most, patients experience anxiety and exhibit symptoms of stress when faced with hypodermic injections via the IM or IV routes. Further, most people have some familiarity with nasal sprays in the form of over-the-counter decongestants for alleviating the symptoms of colds and allergies that they or a family member have used routinely. Another important consideration is that the patient can self-administer the prescribed dosage(s) of nasal spray without the need for trained medical personnel.

There are different intranasal benzodiazepine compositions known in the pharmaceutical arts. However, some intranasal benzodiazepine compositions have poor absorption or delayed time to peak plasma concentration, which is not appropriate, for prevention or treatment of some clinical conditions. Other prior art benzodiazepine formulations do not enhance patient compliance. For example, some intranasal midazolam formulations are produced at a pH that often causes nasal irritation and burning.

Based on the above, there is a need for intranasal benzodiazepine compositions with improved properties, such as for example, rapid absorption and time to peak concentration. There is also a need for intranasal compositions that improve patient compliance.

### SUMMARY

In various embodiments, pharmaceutical compositions for intranasal administration to a mammal are provided. The pharmaceutical composition comprises an effective amount of a benzodiazepine or pharmaceutically acceptable salt thereof and a nasal carrier. In various embodiments, the pharmaceutical composition, when administered intranasally, produce a rapid physiological response.

In various embodiments, a pharmaceutical composition is provided for intranasal administration comprising: an effective amount of a benzodiazepine or pharmaceutically acceptable salt thereof; a nasal carrier; and at least one or more sweeteners, flavoring agents, or masking agents or combinations thereof.

In various embodiments, a pharmaceutical composition is provided for intranasal administration to a mammal comprising: an effective amount of midazolam or pharmaceutically acceptable salt thereof, polyethylene glycol, and propylene glycol.

In various embodiments, a method of treating a mammal in need of rapid sedation, anxiolysis, amnesia, or induction of anesthesia is provided comprising intranasally administering to the mammal an effective amount of a pharmaceutical composition comprising midazolam or pharmaceutically acceptable salt thereof; and a nasal carrier; wherein the rapid sedation, anxiolysis, amnesia, or induction of anesthesia occurs within 5 minutes after intranasal administration.

In various embodiments, a method of treating a mammal in need of rapid sedation, anxiolysis, amnesia, or induction of anesthesia is provided comprising intranasally administering to the mammal an effective amount of a pharmaceutical composition comprising midazolam or pharmaceutically acceptable salt thereof; a nasal carrier; and at least one or more sweeteners, flavoring agents, or masking agents or combinations thereof.

In various embodiments, a method of making a pharmaceutical composition for intranasal administration is provided comprising adding at least one or more sweeteners, flavoring agents, or masking agents or combinations thereof to a pharmaceutical composition comprising midazolam or pharmaceutically acceptable salt thereof, and a nasal carrier so as to make the pharmaceutical composition.

For a better understanding of various embodiments, reference is made to the following description taken in conjunction with the examples, the scope of which is set forth in the appended claims.

### BRIEF DESCRIPTION OF THE FIGURES

Preferred embodiments have been chosen for purposes of illustration and description, but are not intended in any way to restrict the scope of the claims. The preferred embodiments are shown in the accompanying figures, wherein:

FIG. 1 is a graphic representation of mean blood plasma concentration (n=12) of midazolam in plasma versus time for three different midazolam compositions over a four-hour period.

FIG. 2 is a graphic representation of mean blood plasma concentration (n=12) of midazolam in plasma versus time for three different midazolam compositions over a twelve-hour period.

FIG. 3 is a graphic representation of mean blood plasma concentration (n=17) of midazolam in plasma versus time for three different midazolam compositions over a four-hour period.

FIG. 4 is a graphic representation of mean blood plasma concentration (n=17) of midazolam in plasma versus time for three different midazolam compositions over a twelve-hour period.

### DETAILED DESCRIPTION

Various embodiments will now be described. These embodiments are presented to aid in an understanding of the claims and are not intended to, and should not be construed to, limit the claims in any way. All alternatives, modifications and equivalents that may become obvious to those of ordinary skill on reading the disclosure are included within the spirit and scope of the claims.

The pharmaceutical composition comprise benzodiazepine or other compounds. Benzodiazepines, as used herein, include but are not limited to alprazolam, brotizolam, chlordiazepoxide, clobazepam, clonazepam, clorazepate, demoxepam, diazepam, estazolam, flurazepam, quazepam, halazepam, lorazepam, midazolam, nitrazepam, nordazapam, oxazepam, prazepam, quazepam, temazepam, triazolam, zolpidem, zaleplon or combinations thereof. Other compounds that have anxiolytic or sedative effects of some benzodiazepines include, for example, zopiclone, zolpidem, abecarnil, and bretazenil.

In various embodiments, the benzodiazepine may be in free form or in pharmaceutically acceptable salt or complex form. Some examples of pharmaceutically acceptable salts of benzodiazepines include those salt-forming acids and bases that do not substantially increase the toxicity of the compound. Some examples of suitable salts include salts of alkali metals such as magnesium, potassium and ammonium. Salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g. p-toluenesulfonic acids, and the like.

In various embodiments, pharmaceutical compositions are provided for intranasal administration comprising midazolam or pharmaceutically acceptable salts thereof. In various embodiments, the pharmaceutical composition comprises midazolam hydrochloride. Midazolam includes 8-chloro-6-(2-fluorophenyl)-1-methyl-4H-Imidazo-[1,5-a][1,4]benzodiazepine, [CAS 59467-70-8]. The molecular weight of midazolam is 325.8.

Midazolam has the molecular formula: C₁₈H₁₃ClFN₃ and exhibits the following general structure:

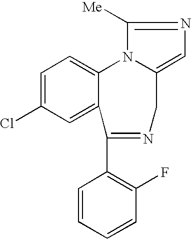

In various embodiments, the pharmaceutical compositions comprise a benzodiazepine or pharmaceutically acceptable salt thereof and a nasal carrier. As used herein, “nasal carrier” includes a solution, emulsion, suspension, or powder designed for delivery of the benzodiazepine or other compound to the nasal mucosa. The nasal carrier may include a diluent suitable for application to the nasal mucosa. Suitable diluents include aqueous or non-aqueous diluents or combinations thereof. Examples of aqueous diluents include, but are not limited to, saline, water, dextrose or combinations thereof. Non-aqueous diluents include, but are not limited to, alcohols, particularly polyhydroxy alcohols such as propylene glycol, polyethylene glycol, glycerol, and vegetable or mineral oils or combinations thereof. These aqueous and/or non-aqueous diluents can be added in various concentrations and combinations to form solutions, suspensions, oil-in-water emulsions or water-in-oil emulsions.

In various embodiments, the nasal carrier comprises polyethylene glycol and propylene glycol. In various embodiments; the polyethylene glycol constitutes from about 15% to about 25% by volume and the propylene glycol constitutes from about 75% to about 85% by volume of the composition. In various embodiments, the polyethylene glycol has an average molecular weight of about 400. In various embodiments, the ratio of polyethylene glycol to propylene glycol is about one to about four.

The nasal carrier, in some embodiments, may also contain excipients such as antioxidants, chemical preservatives, buffering agents, surfactants and/or agents that increase viscosity. Antioxidants are substances that prevent oxidation of the formulations. Suitable antioxidants for use in the pharmaceutical composition, if one is employed, includes but is not limited to, butylated hydroxytoluene, butylated hydroxyanisole, potassium metabisulfite, and the like.

In various embodiments, the composition contains a preservative that is chosen in quantities that preserve the composition, but preferably does not cause irritation to the nasal mucosa. Suitable preservatives for use in some embodiments include, but is not limited to, benzalkonium chloride, methyl, ethyl, propyl or butylparaben, benzyl alcohol, phenylethyl alcohol, benzethonium, or combination thereof. Typically, the preservative is added to the compositions in quantities of from about 0.01% to about 0.5% by weight.

In some embodiments, the formulation is preservative-free. As used herein, preservative-free includes compositions that do not contain any preservative. Thus, the composition does not contain, for example, benzalkonium chloride, methyl, ethyl, propyl or butylparaben, benzyl alcohol, phenylethyl alcohol, or benzethonium.

If a buffering agent is employed in the composition, it is chosen in quantities that preferably do not irritate the nasal mucosa. Buffering agents include agents that reduce pH changes. Some buffering agents that may be used in the pharmaceutical composition include, but are not limited to, salts of citrate, acetate, or phosphate, for example, sodium citrate, sodium acetate, sodium phosphate, and/or combinations thereof. Typically, the buffer is added to the compositions in quantities of from about 0.01% to about 3% by weight.

When one or more surfactants are employed, the amount present in the compositions will vary depending on the particular surfactant chosen, the particular mode of administration (e.g. drop or spray) and the effect desired. In general, however, the amount present will be in the order of from about 0.1 mg/ml to about 10 mg/ml, in various embodiments, about 0.5 mg/ml to 5 mg/ml and, in various embodiments, about 1 mg/ml is used.

In various embodiments, the pharmaceutical composition may include one or more agents that increase viscosity, which are chosen in quantities that preferably do not irritate the nasal mucosa and increase nasal retention time. Some agents that increase viscosity include, but are not limited to, methylcellulose, carboxymethylcellulose sodium, ethylcellulose, carrageenan, carbopol, and/or combinations thereof. In various embodiments, an agent used to increase viscosity and increase nasal retention time is methylcellulose or carbopol. Typically, the agent that increases viscosity may be added to the compositions in quantities of from about 0.1% to about 10% by weight.

To reduce the bitter taste of the intranasal composition and/or enhance patient compliance, in various embodiments, one or more sweeteners or flavoring agents or masking agents are employed. The sweetener or flavoring agent or masking agent includes any agent that sweetens or provides flavor to the pharmaceutical composition: The sweetener or flavoring agent or masking agent will mask the bitter or bad taste that may occur if the pharmaceutical composition drips back into the mouth after intranasal administration. By addition of a sweetener or flavoring agent or masking agent to the intranasal composition, any barrier that a patient may have to taking the intranasal composition because of unpleasant taste is reduced. By adding a sweetener, flavoring agent or masking agent to the intranasal pharmaceutical composition, patient compliance is enhanced or improved.

As used herein, one or more sweeteners or flavoring agents or masking agents include, but are not limited to, acacia syrup, anethole, anise oil, aromatic elixir, benzaldehyde, benzaldehyde elixir, cyclodextrins, compound, caraway, caraway oil, cardamom oil, cardamom seed, cardamom spirit, compound, cardamom tincture, compound, cherry juice, cherry syrup, cinnamon, cinnamon oil, cinnamon water, citric acid, citric acid syrup, clove oil, cocoa, cocoa syrup, coriander oil, dextrose, eriodictyon, eriodictyon fluidextract, eriodictyon syrup, aromatic, ethylacetate, ethyl vanillin, fennel oil, ginger, ginger fluidextract, ginger oleoresin, dextrose, glucose, sugar, maltodextrin, glycerin, glycyrrhiza, glycyrrhiza elixir, glycyrrhiza extract, glycyrrhiza extract pure, glycyrrhiza fluidextract, glycyrrhiza syrup, honey, iso-alcoholic elixir, lavender oil, lemon oil, lemon tincture, mannitol, methyl salicylate, nutmeg oil, orange bitter, elixir, orange bitter, oil, orange flower oil, orange flower water, orange oil, orange peel, bitter, orange peel sweet, tincture, orange spirit, compound, orange syrup, peppermint, peppermint oil, peppermint spirit, peppermint water, phenylethyl alcohol, raspberry juice, raspberry syrup, rosemary oil, rose oil, rose water, rose water, stronger, saccharin, saccharin calcium, saccharin sodium, sarsaparilla syrup, sarsaparilla compound, sorbitol solution, spearmint, spearmint oil, sucrose, sucralose, syrup, thyme oil, tolu balsam, tolu balsam syrup, vanilla, vanilla tincture, vanillin, or wild cherry syrup, or combinations thereof.

In various embodiments, the sweetener is saccharin, sodium saccharin, xylitol, mannitol, glycerin, sorbitol, sucralose, maltodextrin, sucrose, aspartame, acesulfame potassium, dextrose, glycosides, maltose, sweet orange oil, dextrose, glucose, or honey or combinations thereof. Some flavoring agents to use in various embodiments include, but are not limited to, glycerin, wintergreen oil, peppermint oil, peppermint water, peppermint spirit, menthol, or syrup, or combinations thereof. In various embodiments, the masking agents do not make contact with the taste buds. In various embodiments, the masking agent includes, but is not limited to, cyclodextrins, cyclodextrins emulsions, cyclodextrins particles, or cyclodextrin complexes, or combinations thereof.

To reduce burning, if it occurs, the composition may contain an anesthetic agent. Some anesthetic agents include, but are not limited to, lidocaine, prilocaine, procaine, benzocaine tetracaine, chloroprocaine, or pharmaceutically acceptable salts thereof or combinations thereof.

The pharmaceutical compositions, in different embodiments, may also include additional ingredients, such as pharmaceutically acceptable surfactants, co-solvents, adhesives, agents to adjust the pH and osmolarity. The pharmaceutical compositions are not limited to any particular pH. However, generally for nasal administration a mildly acid pH will be preferred. The pH ranges from about 3 to 6 in some embodiments, in other embodiments, pH ranges are from about 3 to about 5, and in other embodiments pH ranges are from about 4 to about 5. If the adjustment of the pH is needed, it can be achieved by the addition of an appropriate acid, such as hydrochloric acid, or base, such as for example, sodium hydroxide.

The pharmaceutical composition in some embodiments can be made, for example, by mixing the benzodiazepine with the nasal carrier and/or a sweetener, flavoring agent, or masking agent or combinations thereof at, for example, room temperature under aseptic conditions to form a mixture. In other embodiments, the mixture is filtered, for example, by a 0.22 micron filter. It will be understood by those of ordinary skill in the art that the order of mixing is not critical, and various embodiments include without limitation mixing of the composition in any order. In various embodiments, the pharmaceutical composition is a sterile solution or suspension.

Pharmaceutical compositions can be administered intranasally by nasal spray, drop, solution, suspension, gel, and the like. Intranasal administration is an art-recognized term and includes, but is not limited to, administration of the composition into the nasal cavity.

When the pharmaceutical composition is a liquid, volumes of the liquid that may be absorbed through the nasal mucosa include, for example, from about 0.025 ml to about 2 ml or from about 0.25 ml to 1 ml, or from about 0.05 ml to about 15 ml in an adult and smaller volumes for children. However, the pharmaceutical compositions are not limited to any one particular volume.

Devices for intranasal delivery are known in the art. Some devices suitable for use with the pharmaceutical compositions are available from, for example, Pfeiffer of America of Princeton, N.J. and Valois of America, Inc. of Greenwich, Conn. These devices are preferred because they have the capability of consistently delivering the pharmaceutical composition. These devices are easily operable by the patient, leave virtually no benzodiazepine remaining in the device after use and can thereafter be discarded without concern that others may abuse the benzodiazepine or other controlled substance.

In various embodiments, the intranasal delivery device may be modified, for example, by increasing the size of the discharge orifice in the nose piece of the applicator to about 0.07 mm for non-aqueous compositions that comprise, for example, polyethylene glycol and/or propylene glycol, in order to accommodate higher viscosity compositions. For aqueous compositions, the diameter can be, for example, from about 0.05 mm in diameter. The intranasal delivery device may also contain a swirl chamber. The applicator components may also be sterilized by methods well known in the art.

The intranasal delivery device may be filled with single or multidose amounts of benzodiazepines. In various embodiments, the device is filled with one single dose of benzodiazepine. In some embodiments, the container holding the pharmaceutical composition and its sealing means are sterilizable, in some embodiments, at least parts of the device that are in contact with the pharmaceutical composition is constructed and assembled in a configuration that can be sterilized. Devices with one or more unit-dose(s) can be sterilized either before or after packaging, employing methods and technology that are well known in the art. Individual devices can be packaged, sterilized and shipped; alternatively, entire shipping and storage packages can be sterilized at once, and the devices removed individually for dispensing, without affecting the sterility of the remaining units.

The amount of benzodiazepine or other compound that can be intranasally administered in accordance with the composition and methods will depend on the particular benzodiazepine chosen, the condition to be treated, the desired frequency of administration and the effect desired. Some medical or veterinary symptoms, syndromes, conditions or diseases that benzodiazepines or other compounds are useful in preventing or treating include, but are not limited to, anxiety, panic attacks, schizophrenia, phobias, sleep disorders (e.g. insomnia) and depressive disorders, agitation, hostility, epilepsy, convulsion, spasticity, involuntary movements, or alcohol withdrawal or combinations thereof. Benzodiazepines or other compounds may be used as adjuncts in medical and dental procedures, such as for example, reducing anxiety before surgical anesthesia, providing sedation, facilitating anesthesia induction, producing amnesia, or to control nausea and vomiting.

In various embodiments, the pharmaceutical composition comprises midazolam and is administered to a mammal in need of rapid sedation, anxiolysis, amnesia, or anesthesia induction. As used herein, an effective amount of benzodiazepine or other compound includes that amount effective to achieve the relief or palliation of symptoms, condition and/or diseases that need benzodiazepine therapy. Maximal dosage of the pharmaceutical composition for a mammal is the highest dosage that elicits the desirable response, which does not cause undesirable or intolerable side effects. The minimal dose of the benzodiazepine is the lowest dose that achieves the desired result. In any event, the practitioner is guided by skill and knowledge in the field, and the present invention includes without limitation dosages that are effective to achieve the desired effect in the mammal. Doses of benzodiazepines suitable for intranasal administration, include but are not limited to, from about 0.1 mg to about 30 mg. For example, doses of midazolam HCL for intranasal administration include, but are not limited to, from about 0.1 mg to about 20 mg.

In various embodiments, it has been surprisingly discovered that pharmaceutical compositions comprising midazolam, when intranasally administered, have rapid absorption and time to peak (Tₘₐₓ) leading to rapid onset than midazolam administered by the IV route. For example, the Tₘₐₓ for intranasally administered midazolam was in some cases about 5 minutes, while the Tₘₐₓ for midazolam administered IV was about 15 minutes. In various embodiments, the pharmaceutical composition comprising midazolam achieves a maximum plasma concentration (Cₘₐₓ) of about 40 ng/mL from a 2.5 mg dose or about 80 ng/mL from a 5 mg dose after intranasal administration. In various embodiments, the ratio of the AUC for intranasal midazolam to AUC of for midazolam after an equivalent dose of intravenous midazolam is at least about 1:1.7.

In various embodiments, the benzodiazepine is administered to a mammal suffering from a condition and/or disease that requires benzodiazepine treatment. Mammals include, for example, humans, as well as pet animals such as dogs and cats, laboratory animals, such as rats and mice, and farm animals, such as horses and cows.

In various embodiments, a method of treating a mammal in need of rapid sedation, anxiolysis, amnesia, or induction of anesthesia is provided. The method comprises intranasally administering to the mammal an effective amount of a pharmaceutical composition comprising midazolam or pharmaceutically acceptable salt thereof in a nasal carrier. The pharmaceutical composition may also contain a sweetener, masking agent or flavoring agent. In various embodiments, the pharmaceutical composition comprising midazolam is intranasally administered to the mammal and the composition is metabolized by the mammal and achieves a 1-hydroxymidazolam plasma level of about 1 to about 8 nanograms/ml.

### EXAMPLES

The examples below demonstrate improved absorption, rapid time to reached peak concentrations, and good bioavailability of the various compositions. The examples also show midazolam compositions that include, for example, sweeteners, which improve patient compliance by reducing the unpleasant taste after intranasal administration.

### Example 1

This example compares 5.0 mg midazolam (MZ) after intranasal (IN), intramuscular (IM) and intravenous (IV) administration in 12 healthy male and female subjects.

### Subjects

Twelve, nonsmoking, healthy subjects (6 male, 6 female) between the ages of 20 and 29 years (mean 22.3 years) and weighing 132 to 202 lbs. (mean 157 lbs.) participated in this inpatient study after giving informed consent. Eleven of the volunteers who enrolled in the study were Caucasian and one was Asian. Study participants were selected based on inclusion/exclusion criteria, medical history, physical and nasal exams, vital signs, laboratory tests, and other procedures as outlined in the protocol. Subjects were within ±20% of ideal body weight in relation to height and elbow breadth and weighed at least 60 kg (132 lbs). The subjects were in good health and had no clinically significant previous nasal surgery or polyps or other physical abnormalities of the nose, cardiovascular, gastrointestinal, renal, hepatic, pulmonary or hematological disease. Subjects who had a history of cerebral trauma with sequelae, hypotension, heart failure, cardiac conduction defect, chronic respiratory disease, bleeding tendency, glaucoma, and a formal diagnosis of sleep apnea or a history of alcohol or substance abuse were excluded. Subjects abstained from alcohol and caffeine containing beverages 48 hours before the dosing period and during the study. Subjects were asked to abstain from prescription and non-prescription drugs that might interact with MZ metabolism or nasal physiology from the date of screening until the end of the study. Subjects had to demonstrate their ability to perform the pharmacodynamic (PD) assessments during the screening evaluation. Informed consent was obtained and this study was conducted according to the applicable guidelines for Good Clinical Practice.

### IV and IM Formulations

The intravenous (IV) and intramuscular (IM) solutions were prepared for administration in the University of Kentucky Hospital Investigational Drug Service Pharmacy using commercially available MZ (Versed® Injection by Hoffman-LaRoche). MZ (5 mL of 1.0 mg/mL) sterile solution was diluted to 10 mL with normal saline for a total volume of 10 mL to be infused over 15 minutes. The 5.0 mg IM MZ (1 mL of 5.0 mg/1.0 mL) was administered without dilution.

### IN Formulation of MZ

The 25 mg/mL IN MZ formulation was prepared under GMP conditions in the University of Kentucky College of Pharmacy Center for Pharmaceutical Science and Technology (CPST). The IN formulation comprised midazolam 25 mg; polyethylene glycol 400, USP 0.18 mL; butylated hydroxytoluene, NF 0.10 mg; saccharin powder, NF 1.00 mg; propylene glycol, USP Q.S. to 1.00 mL. The formulation provided 2.5 mg of MZ in 0.1 mL spray from a modified version of the commercially available, single-dose, metered sprayer (unit dose spray pumps, Pfeiffer of America, Princeton, N.J.). Each subject received a single spray in each nostril for a total of 5.0 mg.

### Protocol

An open-label, randomized, three-way crossover study design was used. Treatment assignments were in the random order generated by a statistician. The three treatments were: Treatment A: 5.0 mg (5 mL of 1.0 mg/mL) IV MZ infused over 15 minutes, Treatment B: 5.0 mg intramuscular MZ (5.0 mg/1.0 mL), and Treatment C: 5.0 mg intranasal MZ solution (2.5 mg/100 μL per sprayer). The three treatments were separated by six-day washout periods. PK blood samples were drawn following each dose. MZ (5 mL of 1.0 mg/mL) sterile solution was diluted to 10 mL with normal saline for a total volume of 10 mL and infused over 15 minutes by a nurse using a stopwatch. IN MZ doses were administered by a physician using Pfeiffer modified unit dose sprayers (Pfeiffer of America, Princeton N.J.). The 5.0 mg IM MZ (5.0 mg/1.0 mL) was administered without dilution. Drug administration occurred in the morning following an overnight fast of at least 8 hours. The subjects continued to fast for 2 hours after dosing. Water was allowed except within two hours before or after drug administration. Subjects were allowed juice, 360 mL, at least 2 hours prior to dosing for each dose. Subjects were awakened 1 hour prior to dosing for performance of PD testing. Blood samples were collected in 10 mL Vacutainer® tubes containing the anticoagulant sodium heparin. Serial blood samples were obtained by venipuncture according to the following schedule: 0 (pre-dose), 5, 10, 20, 30, and 45 minutes, and 1, 1.5, 2, 3, 4, 8, and 12 hours following MZ administration. Actual sampling times were used in PK analysis. After collection, the blood was centrifuged in a refrigerated centrifuge at 4° C. to separate the plasma and the cells, and the plasma was transferred to polypropylene tubes. The plasma was stored at or below −20° C. at the study site until shipped to Kansas City Analytical Services, Inc. (KCAS) in Shawnee, Kans.

LC/MS/MS Assay for MZ and α-hydroxymidazolam

The sample analysis was conducted for MZ and α-hydroxymidazolam using a PE/Sciex API III+LC/MS/MS system in MRM mode by KCAS in Shawnee, Kans. Concentrations less than 0.50 ng/mL were reported as below quantitation limit (BQL). Samples with concentrations greater than 500 ng/mL were reanalyzed using a dilution so that the assayed concentration was within the range of 0.50 to 500.0 ng/mL.

### Pharmacokinetic (PK) Data Analysis

PK parameters were determined using standard noncompartmental methods with log-linear least square regression analysis to determine the elimination rate constants (WinNonlin, Pharsight Corp., Palo Alto, Calif.). The areas under the concentration versus time curves from time zero to infinity (AUC_{0-∞}) were calculated using a combination of the linear and logarithmic trapezoidal rules, with extrapolation to infinity by dividing the last measurable serum concentration by the elimination rate constant (λ_{z}) (Proost, 1985). Values for the maximum concentration (Cₘₐₓ) and time to Cₘₐₓ (Tₘₐₓ) were determined by WinNonlin. The elimination half-life was determined from 0.693/λ_{z}. Clearance (CL/F) was determined by dividing the dose by AUC_{0-∞}. Volumes of distribution for elimination (V_{z}/F) and at steady state (Vₛₛ) were determined by moment curves (Gibaldi and Perrier, 1982). V_{Z}/F was calculated as Dose/(λ_{z}*AUC_{0-∞}). Vₛₛ was calculated as CL*MRT for IV data. The absolute bioavailability (F) for the IN and IM dosage forms was determined by F=AUC_{IN,0-∞}/AUC_{IV,0-∞}. and F=AUC_{IM,0-∞}/AUC_{IV,0-∞}, respectively. Relative bioavailability of the IN compared to the IM dose was calculated by AUC_{IN,0-∞}/AUC_{IM,0-∞}. Mean plasma concentrations were calculated for graphical evaluation only. The calculations included data from samples with measurable concentrations drawn within 5% of the expected sampling time.

### Statistical Data Analysis

Statistical analyses were performed with Statistical Analysis System PC-SAS version 6.12. The statistical tests were 2-sided with a critical level of 0.05. An analysis of variance (ANOVA) with factors sequence, subject (sequence), treatment and period was performed for log-transformed AUC and Cₘₐₓ. The least square geometric means from the ANOVA were used to calculate the ratios and their 90% confidence intervals between treatment groups for AUC and Cₘₐₓ. The carryover effect for the three treatments was analyzed using an ANOVA of log-transformed AUC and Cₘₐₓ. The difference in Tₘₐₓ values between the IN and IM treatments was compared using an ANOVA of rank-transformed Tₘₐₓ. The ANOVA model included factors sequence, subject (sequence), treatment and period. The gender effect for all three treatments was analyzed using an ANOVA of log-transformed AUC and Cₘₐₓ with factors gender, treatment and period.

### Results of Example 1

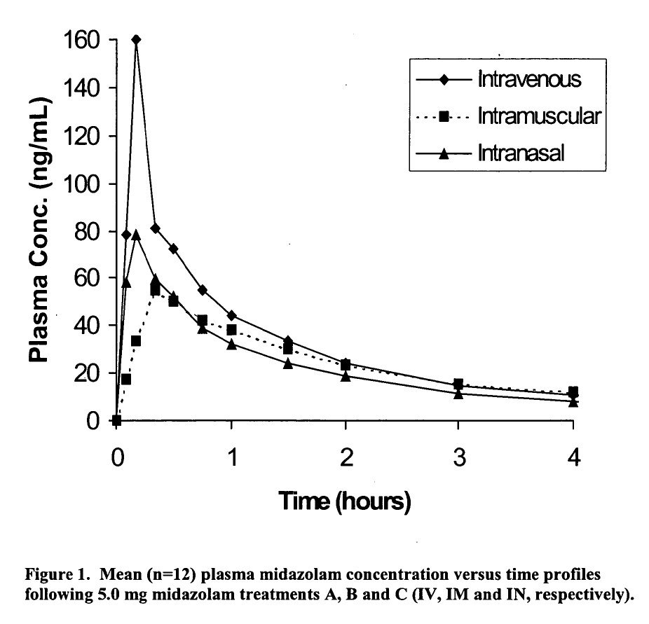

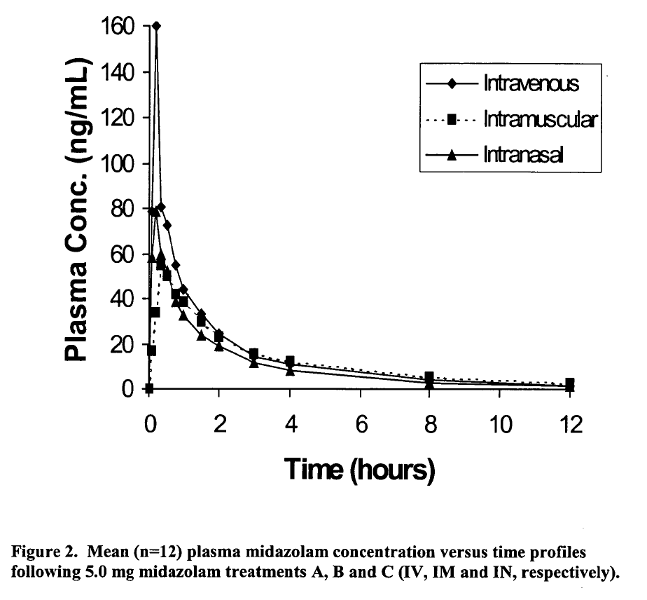

Twelve subjects completed the study without clinically significant or serious adverse events. There were no clinically relevant changes in physical examination, nasal evaluations, or laboratory tests. The principal investigator's review of the data indicated that, in general, doses of the study drug were well tolerated and events were mild to moderate and temporary (2-90 minutes). Two of twelve subjects noted mild dizziness that lasted 35 and 50 minutes. Three of twelve subjects noted blurred vision that lasted 5-90 minutes. No subjects experienced respiratory depression, apnea, laryngospasm, bronchospasm or wheezing. The mean plasma concentration versus time curve profiles over the first 4 hours and the entire 12 hours for the three doses are shown in FIGS. 1 and 2. FIG. 1 shows that absorption of MZ following IN administration was very rapid. MZ concentrations reached a peak in 2 individuals at 5 min and in 8 of 12 individuals in 10 min or less. No secondary or late bumps indicating absorption from swallowing the IN dose were observed in the plasma concentration time curves. Table 1 summarizes PK data for the three treatments. Median Tₘₐₓ values were 10 and 30 min for the IN and IM doses, respectively. Cₘₐₓ values after the IN dose were higher than those after the IM dose and occurred consistently earlier. Relative bioavailability of the IM to IN dose was on average 79%. Unfortunately, the absolute bioavailability of MZ by the IN and IM routes in Table 1 is overestimated due to the underestimation of the AUC_{O-∞} for the IV dose. The AUC_{0-∞} given for the IV dose underestimates the true AUC_{0-∞} because the area around the Cₘₐₓ (which would have been at the end of the 15 minute infusion) was not captured in this study. However, the data for the IM dose are accurate and acceptable for making conclusions regarding the relative bioavailability of the IN dose compared to the IM dose. The high relative bioavailability of the IN to IM dose confirms that bioavailability was good for MZ administered by the IN route.

TABLE 1
Mean (CV as a %) Single Dose MZ Pharmacokinetic (PK) Parameters
Following Administration of 5.0 mg Intravenous (IV), Intramuscular
(IM) and Intranasal (IN) MZ in Healthy Subjects (n = 12)

| 1.1. | IV | IM | IN |
|---|---|---|---|
| PK Parameter | (5.0 mg) | (5.0 mg) | (5.0 mg) |
| Tₘₐₓ (min)* | 10 (5-31) | 30 (20-60) | 10 (5-20) |
| Cₘₐₓ (ng/mL) | 167.3 (28.9) | 58.7 (49.7) | 80.0 (20.8) |
| t_{1/2} (hr) | 3.14 (23.0) | 4.17 (50.2) | 3.25 (29.8) |
| AUC₀₋ₜ (ng · hr/mL) | 178.1 (17.1) | 152.3 (25.8) | 126.7 (20.6) |
| AUC_{0-∞} (ng · hr/mL) | 186.4 (16.5) | 174.6 (22.1) | 133.8 (19.4) |
| MRT (hr) | 2.88 (20.2) | 5.48 (48.9) | 3.33 (27.4) |
| CL/F or CLₛₛ/F (L/hr) | 27.5 (17.8) | 30.1 (24.6) | 38.6 (19.2) |
| Vₛₛ (L) | 78.8 (23.3) | — | — |
| V_{z}/F (L) | 123.4 (26.1) | 177.9 (51.7) | 182.3 (39.0) |
| F (%)** | assume 100% | 93.4 (12.4) | 72.5 (16.8) |
| Relative F (IM/IN) | — | — | 79.2 (23.7) |
| (%) | | | |

*median and range given for Tₘₐₓ;
**see above for discussion of F.-

No significant gender differences were found for AUC_{0-∞} and Cₘₐₓ values (P>0.1). The gender effect was significant for AUC₀₋ₜ values (P=0.0452, M>F). Larger differences in AUC₀₋ₜ between males and females were observed for the IM formulation. The differences were smaller for the IN formulation (12%). Data were combined for analysis of treatment effects. A significantly shorter Tₘₐₓ was observed for the IN formulation compared to the IM formulation (p=0.0001). Tₘₐₓ and Cₘₐₓ were not captured at the end of the infusion for the IV dose. Statistical analysis of carryover effect on log transformed AUC_{0-∞}, AUC₀₋ₜ and Cₘₐₓ for the two IN treatments was performed. P-values from an ANOVA with factors sequence, subject (sequence), treatment and period for sequence BC and CB were >0.1, so the carryover effects were not significant and this implies the validity of the analyses in Table 2.

Table 2 summarizes the ratios and 90% confidence intervals (CI) of Cₘₐₓ and AUCs after Treatments A, B and C. AUC₀₋ₜ and AUC₀₋ₒₒ were more comparable between the IM and IV treatments (B/A) than between the IV and IN (C/A) treatments. However, Cₘₐₓ values were almost 50% higher after Treatment C (IN) compared to Treatment B (IM).

TABLE 2
Summary of Ratios of Least Squares Geometric Means and 90% Confidence
Intervals

| | 1.1.2. Treatment | | | | | |
|---|---|---|---|---|---|---|
| | Group | | | B/A | C/A | C/B |
| | Geometric Means | | | (IM/IV) | (IN/IV) | (IN/IM) |
| | 5 mg MZ | 5 mg MZ | 5 mg MZ | Ratio | Ratio | Ratio |
| Parameter | IV (A) | IM (B) | IN (C) | (90% CI) | (90% CI) | (90% CI) |
| AUC_{0-∞} | 184.01 | 170.51 | 131.58 | 0.93 | 0.72 | 0.77 |
| (ng · hr/mL) | | | | (0.85-1.01) | (0.65-0.78) | (0.71-0.84) |
| AUC₀₋ₜ | 175.72 | 147.81 | 124.29 | 0.84 | 0.71 | 0.84 |
| (ng · hr/mL) | | | | (0.77-0.92) | (0.65-0.77) | (0.77-0.92) |
| Cₘₐₓ | 159.02 | 53.28 | 78.35 | 0.34 | 0.49 | 1.47 |
| (ng/mL) | | | | (0.26-0.43) | (0.38-0.63) | (1.15-1.88) |

CI = Confidence Intervals
Least squares geometric means are from an ANOVA with with factors sequence, subject(sequence), treatment and period for log-transformed AUCs and Cₘₐₓ.

The 1-hydroxymidazolam metabolite concentrations were consistently lower than those of the parent drug.

### Discussion

The pharmacokinetics of MZ were evaluated in 12 healthy male and female volunteers after single 5.0 mg doses of N, IM and IN MZ. All subjects completed the study without clinically significant or serious adverse events. The pharmacokinetics of MZ were consistent with rapid but relatively short duration of action. The mean absolute bioavailability of IN MZ would be predicted to be around 65% assuming that about 7% of the IV AUC was missed. The mean relative bioavailability compared to the IM dose was 79%. Less than complete bioavailability after the IN administration may be explained by metabolism during absorption across the nasal mucosa or simply incomplete absorption and swallowing. There was no evidence of swallowing. Plasma clearance and volumes of distribution were high. The IN formulation of MZ had rapid absorption (median peak times of 10 min). In comparison with IM administration, the IN formulation had earlier and higher peak plasma concentrations.

### CONCLUSION

Intravenously administered MZ distributes extensively and rapidly in the body. A total systemic clearance of 28 L/hr indicates that MZ is a highly cleared drug. The IN formulation of MZ had rapid absorption and reached peak concentrations significantly more rapidly than the IM dose. Absolute bioavailability of MZ from the IN dosage form was good and supports further investigation of this dosage form for clinical use. Relative bioavailability compared to the IM dose was 79.2% (23.7% CV). No treatment emergent adverse events were observed during the conduct of this protocol that would preclude further study of MZ in healthy subjects. Adverse events were mild and expected for this drug. As evidenced by the lack of cardiovascular and respiratory adverse events, all the subjects tolerated the drug well.

### Example 2

This study compares the pharmacokinetics of midazolam (MZ) after administration of 2.5 and 5.0 mg intranasal (IN) MZ and 2.5 mg intravenous (IV) MZ in 18 healthy male and female subjects.

### Subjects

Eighteen, nonsmoking, healthy subjects (9 male, 9 female) between the ages of 20 and 29 years (mean 22.3 years) and weighing 60 to 92 kg (mean 71 kg) participated in this inpatient study after giving informed consent. Seventeen of the volunteers who enrolled in the study were Caucasian and one was African-American. Seventeen subjects completed the study. Study participants were selected based on inclusion/exclusion criteria, medical history, physical and nasal exams, vital signs, laboratory tests, and other procedures as outlined in the protocol. Subjects were within ±25% of ideal body weight in relation to height and elbow breadth and weighed at least 60 kg (132 lbs). The subjects were in good health, between 18 and 45 years of age and had no clinically significant previous nasal surgery or polyps or other physical abnormalities of the nose, vital signs, cardiovascular, gastrointestinal, renal, hepatic, pulmonary, hematological or neurological disease. Subjects who had a history of a seizure disorder, cerebral trauma with sequelae, hypotension, heart failure, cardiac conduction defect, chronic respiratory disease, bleeding tendency, narrow-angle glaucoma, a formal diagnosis of sleep apnea, a current formal diagnosis of depressive disorder or psychosis or a medical diagnosis of alcohol or substance abuse were excluded. Subjects with a known history of Gilbert's Syndrome or with any other etiology for an increased serum total bilirubin level and subjects with any other clinical condition that might affect the absorption, distribution, biotransformation, or excretion of the drug (e.g., acute respiratory illness, allergic rhinitis, etc.) or were allergic to MZ or formulation components were excluded. Subjects who had a history of regular sedative/hypnotic medication use (i.e., at least once per week) or who had taken any sedative/hypnotic medications within the 2 weeks prior to study drug administration were excluded. Subjects abstained from alcohol and caffeine containing beverages 48 hours before the dosing period and during the study. Subjects were asked to abstain from prescription and non-prescription medication, vaccines, herbal and nutritional supplements that might interact with MZ metabolism or nasal physiology within 7 days of dosing and during the study.

### IV Formulation

The intravenous (IV) solutions were prepared for administration in the University of Kentucky Hospital Investigational Drug Service Pharmacy using commercially available MZ (Versed® Injection by Hoffman-LaRoche). MZ (0.5 mL of 5.0 mg/mL) sterile solution was diluted to 10 mL with normal saline for a total volume of 10 mL to be infused over 15 minutes.

### IN Formulation of MZ

The 25 mg/mL IN MZ formulation was prepared under GMP conditions in the University of Kentucky College of Pharmacy Center for Pharmaceutical Science and Technology (CPST). The IN formulation contained midazolam 25 mg; polyethylene glycol 400, USP 0.18 mL; butylated hydroxytoluene, NF 0.10 mg; saccharin powder, NF 1.00 mg; propylene glycol, USP Q.S. to 1.00 mL. The formulation provided 2.5 mg of MZ in 0.1 mL spray from a modified version of the commercially available, single-dose, metered sprayer (unit dose spray pumps, Pfeiffer of America, Princeton, N.J.). Each subject received a single spray in one nostril for a 2.5 mg dose or a single spray in each nostril for a total of 5.0 mg.

### Protocol

An open-label, randomized, three-way crossover study design was used. Treatment assignments were in the random order generated by a statistician. The three treatments were: Treatment A: 2.5 mg (5 mL of 1.0 mg/mL) IV MZ infused over 15 minutes, Treatment B: 2.5 mg intranasal MZ solution, one 2.5 mg/100 μL sprayer, and Treatment C: 5.0 mg intranasal MZ solution, two 2.5 mg/100 μL sprayers, one sprayer per naris. The three treatments were separated by six-day washout periods. PK blood samples were drawn following each dose. MZ (5 mL of 1.0 mg/mL) sterile solution was diluted to 10 mL with normal saline for a total volume of 10 mL and infused over 15 minutes by a nurse using a stopwatch. IN MZ doses were administered by a physician using Pfeiffer modified unit dose sprayers (Pfeiffer of America, Princeton N.J.). Drug administration occurred in the morning following an overnight fast of at least 8 hours. The subjects continued to fast for 2 hours after dosing. Water was allowed except within two hours before or after drug administration. Subjects were allowed juice, 240 mL, at least 2 hours prior to dosing for each dose. Grapefruit juice was not allowed during the study. Blood samples were collected in 10 mL Vacutainer® tubes containing the anticoagulant sodium heparin. Serial blood samples were obtained by venipuncture according to the following schedule: 0 (pre-dose), 5, 10, 15, 20, 30, and 45 minutes, and 1, 1.5, 2, 3, 4, 8, and 12 hours following MZ administration. Actual sampling times were used in PK analysis. After collection, the blood was centrifuged in a refrigerated centrifuge at 4° C. to separate the plasma and the cells, and the plasma was transferred to polypropylene tubes. The plasma was stored at or below −20° C. at the study site until shipped to Kansas City Analytical Services, Inc. (KCAS) in Shawnee, Kans.

LC/MS/MS Assay for MZ and α-hydroxymidazolam

The sample analysis was conducted for MZ and α-hydroxymidazolam using a PE/Sciex API III+LC/MS/MS system in MRM mode by KCAS in Shawnee, Kans. Concentrations less than 0.50 ng/mL were reported as below quantitation limit (BQL). Samples with concentrations greater than 500 ng/mL were reanalyzed using a dilution so that the assayed concentration was within the range of 0.50 to 500.0 ng/mL.

### Pharmacokinetic (PK) Data Analysis

IN doses were determined by weighing the nasal spray pumps before and after dosing. These weights and the concentrations of the IN solutions (2.5 mg/mL, density 1.056) were used to confirm each subject's dose and to evaluate delivery. The dose weights were not used for PK analysis. PK parameters were determined using standard noncompartmental methods with log-linear least square regression analysis to determine the elimination rate constants (WinNonlin, Pharsight Corp., Palo Alto, Calif.). The areas under the concentration versus time curves from time zero to infinity (AUC_{0-∞}) were calculated using a combination of the linear and logarithmic trapezoidal rules, with extrapolation to infinity by dividing the last measurable serum concentration by the elimination rate constant (λ_{z}) (Proost, 1985). Values for the maximum concentration (Cₘₐₓ) and time to Cₘₐₓ (Tₘₐₓ) were determined by WinNonlin. The elimination half-life was determined from 0.693/λ_{z}. Clearance (CL/F) was determined by dividing the dose by AUC_{0-∞}. Volumes of distribution for elimination (V_{z}/F) and at steady state (Vₛₛ) were determined by moment curves (Gibaldi and Perrier, 1982). V_{z}/F was calculated as Dose/(λ_{z}*AUC_{0-∞}). Vₛₛ was calculated as CL*MRT for IV data. The absolute bioavailability (F) for the IN dosage form was determined by F=AUC_{IN,0-∞}/AUC_{IV,0-∞}. Mean plasma concentrations were calculated for graphical evaluation only. The calculations included data from samples with measurable concentrations drawn within 5% of the expected sampling time.

### Pharmacodynamic (PD) Data Analysis

Self-report measures were collected using Visual Analog Scales (VAS) and the Stanford Sleepiness Scale (SSS). The VAS and SSS were administered at 0 (pre-dose), 10, 20, 30, and 45 minutes, and 1, 1.5, 2, 3, 4, 6, 8, and 12 hours after initiation of the IV dose and administration of the IN doses. Observer Sedation Rating was also performed. The observer for each subject rated the degree of sedation using a qualitative categorical measure of sedation at 0 (pre-dose), 5, 10, 20, 30, and 45 minutes, and 1, 1.5, 2, 3, 4, 6, 8, and 12 hours after initiation of the IV dose and administration of the IN doses. The Observer's Assessment of Alertness/Sedation Scale was used to rate sedation at the above time points. The OAA/S Scale is composed of the following categories: responsiveness, speech, facial expression, and eyes. Subjects were evaluated in each category. The OAA/S was scored in two ways. A composite score was documented as the lowest score in any one of the four assessment categories. A sum score was calculated as the sum of the four category scores. Dependent variables were analyzed as a function of treatment. Analyses of peak effects, time to peak effects, and AUCs, using linear trapezoidal rules, were also evaluated. Separate AUC analyses were completed for AUC between baseline and 4 hours after dose (AUC4, over the duration of peak effects) as well as between baseline and last measurable point and 12 hours after dose (AUCa11 and AUC12, respectively).

### Statistical Data Analysis

Statistical analyses were performed with PC-SAS (version 6.12, SAS Institute, Cary, N.C.). The statistical tests for PK parameters were 2-sided with a critical level of 0.05 unless specified otherwise. An analysis of variance (ANOVA) with factors sequence, subject (sequence), treatment and period was performed for log-transformed AUC and Cₘₐₓ. The least square geometric means from the ANOVA were used to calculate the ratios and their 90% confidence intervals between treatment groups for AUC and Cₘₐₓ. The carryover effect for the three treatments was also assessed using the ANOVA. The gender effect for all three treatments was analyzed using an ANOVA of log-transformed AUC and Cₘₐₓ with factors gender, treatment and period. One subject 216's data for Treatment B was included in the summary statistics of PK parameters. However, Subjects 216 (with outlier for Treatment B) and 218 (early withdrawal) were excluded from the PK analyses for evaluable subjects.

Effects of treatment on each PD parameter were tested using ANOVA with factors sequence, subject (sequence), treatment and period. The carryover effects for the treatment PD effects were also assessed using ANOVA. In some cases, significant carryover was found but this was expected because repetition of tests has been shown to produce performance changes.

### PK Results of Example 2

Seventeen subjects completed the study without clinically significant or serious adverse events. One subject received a single 2.5 mg IN dose and then did not return for subsequent treatments. There were no clinically relevant changes in physical examination, nasal evaluations, or laboratory tests. The principal investigator's review of the data indicated that, in general, doses of the study drug were well tolerated and events were mild to moderate and temporary. There were 1, 2 and no reports of dizziness after the 2.5 mg IV, 2.5 mg IN and 5.0 mg IN doses, respectively. Dizziness lasted up to 86 minutes. Three out of eighteen subjects noted blurred or double vision that lasted 5-40 minutes. No subjects experienced respiratory depression, apnea, laryngospasm, bronchospasm or wheezing.

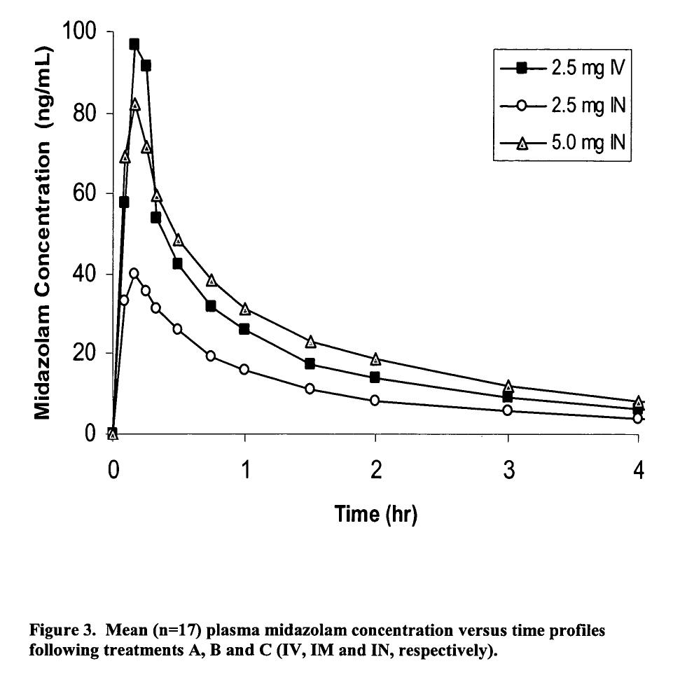

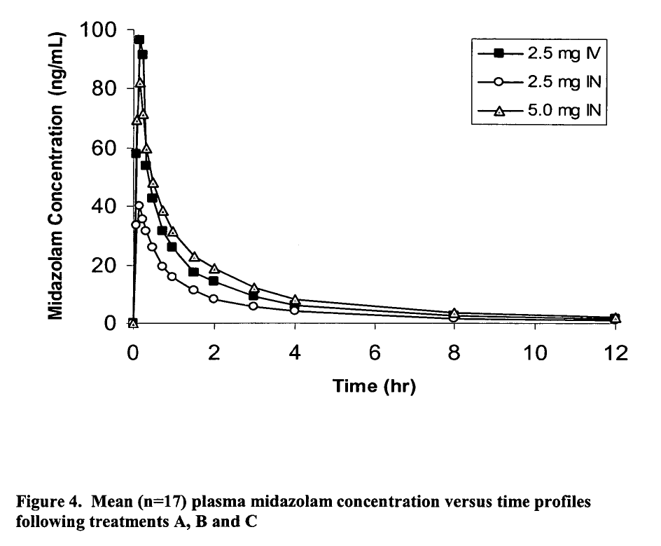

The mean plasma concentration versus time curve profiles over the first 4 hours and the entire 12 hours for the three treatments are shown in FIGS. 3 and 4. FIG. 3 shows that the absorption of MZ following IN administration was very rapid.

MZ concentrations reached a peak at 5 min in one-quarter to one-third of the individuals for the two IN treatments. Median Tₘₐₓ values were 10 min (range 5 to 20 min) for the 2.5 mg and 5.0 mg IN doses. Three individuals had Cₘₐₓ values after the 5.0 mg IN dose that were higher than the Cₘₐₓ after the 15 minute, 2.5 mg IV infusion. One subject had plasma concentrations that were low and they increased and decreased with no pattern. His elimination rate constant was indeterminant as a result. The concentrations ranged from 1.15 to 3.16 ng/mL over the 4 hour period and then dropped to below quantifiable limits.

Table 3 summarizes PK data for the three treatments. Tₘₐₓ values were not significantly different for the two IN treatments (P>0.2).

TABLE 3
Mean (CV as a %) Single Dose MZ Pharmacokinetic (PK) Parameters
Following Administration of Intravenous (IV) and Intranasal (IN) MZ
in Healthy Subjects

| 1.2. | Treatment A | Treatment B | Treatment C |
|---|---|---|---|
| PK Parameter | 2.5 mg IV | 2.5 mg IN | 5.0 mg IN |
| Tₘₐₓ (min)* | 15 (10-15) | 10 (5-20) | 10 (5-20) |
| Cₘₐₓ (ng/mL) | 108.5 (13.5) | 44.5 (38.4) | 83.9 (28.9) |
| t_{1/2} (hr) | 4.03 (33.8) | 4.00 (33.4) | 4.07 (34.2) |
| AUC₀₋ₜ (ng · hr/mL) | 109.2 (12.1) | 65.8 (31.9) | 130.9 (24.7) |
| AUC_{0-∞} (ng · hr/mL) | 119.3 (14.1) | 72.6 (30.6)) | 143.6 (24.5) |
| MRT (hr) | 3.70 (31.7) | 4.18 (33.8) | 4.18 (28.3) |
| CL or CL/F (L/hr) | 21.4 (14.3) | 43.9 (93.9) | 37.0 (26.6) |
| Vₛₛ (L) | 77.3 (25.2) | — | — |

The actual doses administered presented were determined by weighing the pumps before and after dosing. They were lower that the intended doses, on average, by about 16% (Table 4). The range was from 38% below to 20% above the intended dose.

TABLE 4
Mean (CV as a %) Dose Weights Following Administration of
Intranasal (IN) MZ in Healthy Subjects

| 1.3.IN [symbol] | | | | | | |
|---|---|---|---|---|---|---|
| Dose | N | Mean | % CV | Min | Max | % of Dose |
| 2.5 mg | 16 | 2.09 | 12.9 | 1.60 | 2.50 | 83.7 |
| 5.0 mg | 17 | 4.22 | 7.98 | 3.77 | 5.21 | 84.4 |

[symbol]

indicates data missing or illegible when filed

Absolute bioavailability of the MZ was, on average, 60-61% for the IN doses. However, the absolute bioavailability of MZ by the IN routes in Table 3 is underestimated due to the less than expected dose delivery of the nasal sprayers. The dose weight data that are given in Table 4 show that on average, the delivered dose in this study was about 84% of the planned dose. Recalculating the bioavailability based on the actual doses administered (by weight) would make the bioavailability about 72% for the IN doses. No significant gender differences were found for AUC_{0-∞} and Cₘₐₓ values (P>0.1). The gender effect was significant for dose-normalized AUC₀₋ₜ values (P=0.0371, M>F). Data were combined for analysis of treatment effects. Statistical analysis of carryover effect on log transformed AUC_{0-∞}, AUC₀₋ₜ and Cₘₐₓ for the two IN treatments was performed. P-values from an ANOVA with factors sequence, subject (sequence), treatment and period for sequence were >0.3, so the carryover effects were not significant and this implies the validity of the analyses in Table 5.

Table 5 summarizes the ratios and 90% confidence intervals (CI) of Cₘₐₓ and AUCs after Treatments A, B and C. The ratio of dose normalized Cₘₐₓ and AUC values were near unity after Treatment C (IN) compared to Treatment B (IN), as expected.

TABLE 5
Summary of Ratios of Least Squares Geometric Means and 90%
Confidence Intervals
(Dose Normalized Parameters)

| | Treatment Group | | | | | |
|---|---|---|---|---|---|---|
| | Geometric Means | | | B/A | C/A | C/B |
| | 2.5 mg | 2.5 mg | 5.0 mg | (IN/IV) | (IN/IV) | (IN/IV) |
| | MZ IV | MZ IN | MZ IN | Ratio | Ratio | Ratio |
| Parameter | (A) | (B) | (C) | (90% CI) | (90% CI) | (90% CI) |
| AUC_{0-∞} | 47.80 | 29.13 | 28.42 | 0.61 | 0.59 | 0.98 |
| (ng · hr/mL) | | | | (0.54-0.69) | (0.52-0.67) | (0.86-1.11) |
| AUC₀₋ₜ | 43.67 | 26.31 | 25.75 | 0.60 | 0.59 | 0.98 |
| (ng · hr/mL) | | | | (0.53-0.68) | (0.52-0.67) | (0.86-1.11 |
| Cₘₐₓ | 42.15 | 17.12 | 15.93 | 0.41 | 0.38 | 0.93 |
| (ng/mL) | | | | (0.34-0.48) | (0.32-0.45) | (0.78-1.11) |

CI = Confidence Intervals
Log-transformed data are analyzed using an ANOVA with factors sequence, subject(sequence), treatment and period.
Dose normalized data are used (2.5 or 5.0 mg).

The α-hydroxymidazolam metabolite concentrations were consistently lower than those of the parent drug.

### PD Results of Example 2

Table 6 summarizes analyses of PD VAS ratings. Cₘₐₓ (peak effects), time to peak effects (Tmax), and areas under the ratings curves are given (AUC4, AUC12 and AUCa11) for the VAS ratings. VAS parameters that showed statistical significance and their P values are listed in alphabetical order above the break in Table 6. These ratings illustrate the typical effects of dose and route on MZ PD. On 30 out of 40 measures, the order of magnitude of effects were identical with IV producing the greatest effects followed by the higher IN dose and then the lower IN dose. There were many trends in these data, however, only ratings of 10 parameters out of 40 reached significance. No differences were obtained on Tmax. No parameters for “willing to take drug again,” “anxious” or “stimulated” reached significance. Due to the large number of missing values, the results from VAS ratings should be interpreted with caution. These statistical comparisons are presented for their usefulness in future study design.

TABLE 6
Mean (SD) midazolam PD parameters following Treatments A, B and C

| | | | | | |
|---|---|---|---|---|---|
| | Variable | 2.5 mg MZ IV | 2.5 mg MZ IN | 5.0 mg MZ IN | |
| Parameter | Name | Treatment A | Treatment B | Treatment C | P Value |

P < 0.05

| | | | | | | | | |
|---|---|---|---|---|---|---|---|---|
| fatigue | AUC12 | 158.88 | (149.98) | 75.24 | (58.97) | 108.71 | (76.91) | 0.0213 |
| fatigue | AUC4 | 86.24 | (53.67) | 48.47 | (38.88) | 72.46 | (46.95) | 0.0054 |
| fatigue | AUCall | 140.83 | (137.18) | 78.79 | (57.56) | 99.45 | (71.32) | 0.0200 |
| fatigue | Cmax | 53.59 | (22.17) | 36.72 | (21.14) | 48.29 | (20.68) | 0.0080 |
| Feel | AUC12 | 87.58 | (53.23) | 64.85 | (44.60) | 95.54 | (54.92) | 0.0430 |
| Feel | AUC4 | 64.88 | (33.38) | 48.05 | (34.88) | 75.37 | (47.54) | 0.0211 |
| Feel | Cmax | 56.06 | (17.52) | 40.22 | (26.43) | 59.41 | (21.11) | 0.0085 |
| High | Cmax | 46.35 | (26.07) | 27.39 | (18.08) | 38.53 | (22.61) | 0.0053 |
| Like | Cmax | 61.31 | (22.98) | 47.38 | (22.75) | 70.00 | (19.47) | 0.0264 |
| Sedate | Cmax | 55.85 | (19.27) | 40.22 | (22.70) | 52.35 | (13.60) | 0.0157 |

P > 0.05

| | | | | | | | | |
|---|---|---|---|---|---|---|---|---|
| anxious | AUC12 | 54.79 | (66.56) | 46.18 | (72.33) | 54.76 | (84.30) | 0.4220 |
| anxious | AUC4 | 29.78 | (28.52) | 18.64 | (17.40) | 25.95 | (32.12) | 0.0849 |
| anxious | AUCall | 53.76 | (61.44) | 49.71 | (72.20) | 52.94 | (77.28) | 0.2931 |
| anxious | Cmax | 26.79 | (25.29) | 15.36 | (15.71) | 19.26 | (20.76) | 0.1023 |
| anxious | Tmax | 0.51 | (0.56) | 1.52 | (2.89) | 2.41 | (3.55) | 0.0978 |
| fatigue | Tmax | 0.78 | (0.50) | 1.10 | (1.58) | 1.11 | (1.44) | 0.6626 |
| Feel | AUCall | 86.43 | (53.96) | 68.76 | (46.02) | 86.98 | (54.25) | 0.0646 |
| Feel | Tmax | 0.72 | (0.52) | 0.74 | (0.92) | 0.69 | (0.50) | 0.9469 |
| High | AUC12 | 66.81 | (38.03) | 54.74 | (42.79) | 62.24 | (51.11) | 0.2549 |
| High | AUC4 | 48.28 | (30.67) | 32.82 | (25.47) | 45.52 | (38.64) | 0.1299 |
| High | AUCall | 66.33 | (41.50) | 58.14 | (45.62) | 61.94 | (49.98) | 0.3256 |
| High | Tmax | 1.06 | (2.85) | 0.54 | (0.69) | 1.40 | (2.83) | 0.5662 |
| Like | AUC12 | 339.54 | (321.48) | 270.93 | (290.84) | 309.07 | (234.08) | 0.6696 |
| Like | AUC4 | 126.94 | (78.08) | 106.78 | (86.06) | 119.62 | (64.44) | 0.6350 |
| Like | AUCall | 288.98 | (293.37) | 246.16 | (268.39) | 253.80 | (224.50) | 0.8362 |
| Like | Tmax | 2.52 | (3.01) | 1.08 | (1.83) | 2.07 | (2.81) | 0.2344 |
| Sedate | AUC12 | 95.76 | (79.62) | 68.43 | (56.69) | 99.77 | (68.40) | 0.0702 |
| Sedate | AUC4 | 71.42 | (46.70) | 52.29 | (45.44) | 70.24 | (40.68) | 0.1273 |
| Sedate | AUCall | 93.47 | (73.41) | 73.15 | (55.43) | 92.02 | (64.32) | 0.0931 |
| Sedate | Tmax | 0.75 | (0.50) | 0.53 | (0.47) | 0.62 | (0.37) | 0.2946 |
| Stim | AUC12 | 172.23 | (195.40) | 148.22 | (182.23) | 187.20 | (201.87) | 0.3108 |
| Stim | AUC4 | 67.07 | (52.47) | 56.53 | (53.38) | 67.09 | (50.51) | 0.5830 |
| Stim | AUCall | 187.40 | (196.21) | 157.54 | (177.94) | 184.74 | (192.46) | 0.5364 |
| Stim | Cmax | 40.00 | (21.78) | 33.36 | (20.50) | 41.41 | (22.03) | 0.3008 |
| Stim | Tmax | 1.62 | (2.90) | 1.74 | (2.86) | 2.69 | (3.93) | 0.3200 |
| Will | AUC12 | 739.92 | (396.94) | 690.63 | (392.61) | 714.86 | (368.68) | 0.5568 |
| Will | AUC4 | 241.10 | (102.12) | 215.94 | (122.09) | 233.52 | (108.09) | 0.6826 |
| Will | AUCall | 699.79 | (385.66) | 638.61 | (400.75) | 704.00 | (379.58) | 0.5389 |
| Will | Cmax | 79.44 | (16.62) | 76.25 | (25.26) | 80.26 | (20.05) | 0.9669 |
| Will | Tmax | 3.09 | (4.06) | 2.73 | (3.37) | 2.76 | (3.90) | 0.9608 |

P values from ANOVA.
Note:
These ratings are not the same as the similarly named PK parameters.
Units for parameters: Tmax (hr), Cmax (rating score), AUC4, AUC12 and AUCall (rating * hour).

### Discussion

The pharmacokinetics of MZ were evaluated in healthy male and female volunteers after single 2.5 mg and 5.0 mg doses of IV and IN MZ. Seventeen out of eighteen subjects completed the study without clinically significant or serious adverse events. One male subject dropped out for scheduling reasons after receiving one treatment. The pharmacokinetics of MZ were consistent with rapid absorption (median peak times of 10 minutes after IN administration). but relatively short duration of action. The mean absolute bioavailability of IN MZ was approximately 60-61%. However, based on actual dose delivery weights, bioavailability was about 72% for the IN doses. The 84% delivery of doses was most likely because of under filling of sprayers during manufacturing. The remainder of the incomplete bioavailability after the IN administration may be explained by metabolism during absorption across the nasal mucosa or simply, incomplete absorption and swallowing. There was no evidence of swallowing but that would be expected due to the low oral bioavailability of MZ. Plasma clearance and volumes of distribution were high, as expected for MZ.

PD analyses indicated clearly that all three treatments produced changes in subjective ratings of sleep scores, VAS ratings and observer ratings. The intensity of the PD effects was greatest over the first 2 hours following dose administration. The order of magnitude of effects on all PD outcome measures were not always identical but in most cases, IV produced the largest or a similar duration/magnitude of effects compared to the high dose of IN MZ which was followed by the low IN MZ dose. The peak time of effects did not differ statistically between IV and IN doses. The onset did not vary with dose as much as the duration of effect did, as determined through the AUC analyses.

### CONCLUSION

Intravenously administered MZ distributes extensively and rapidly in the body. A total systemic clearance of 21 L/hr indicates that MZ is a highly cleared drug. The IN formulation of MZ had rapid absorption with median times of 10 minutes to achieve peak concentrations. The rise in plasma concentrations matched the IV infusion in some cases. The α-hydroxymidazolam metabolite concentrations were consistently lower than those of the parent drug. The absolute bioavailability of MZ from the IN dosage form was approximately 60% and supports further investigation of this dosage form for clinical use. PD analyses indicated clearly that all three treatments produced changes in subjective ratings of sleep scores, VAS ratings and observer ratings. The intensity of the PD effects was greatest over the first 2 hours following dose administration.

No treatment emergent adverse events were observed during the conduct of this protocol that would preclude further study of MZ in healthy subjects. Adverse events were unremarkable and expected for this drug. As evidenced by the lack of cardiovascular and respiratory adverse events, all the subjects tolerated the drug well.

Having now generally described the embodiments, the same may be more readily understood through the following reference to the following example, which is provided by way of illustration and is not intended to limit the present invention unless specified.

What is claimed is:

1-10. (canceled)

11. A sterile, liquid pharmaceutical composition for intranasal administration to a mammal comprising: an effective amount of midazolam or pharmaceutically acceptable salt thereof for inducing rapid sedation, anxiolysis, amnesia or anesthesia when administered intranasally to a mammal, from about 15% to about 25% by volume polyethylene glycol, and propylene glycol; wherein the midazolam achieves a time to maximum plasma concentration (Tₘₐₓ) within about 10 minutes after intranasal administration of the pharmaceutical composition.

12. A pharmaceutical composition according to claim 11, wherein the polyethylene glycol comprises from about 15% to about 25% by volume and the propylene glycol constitutes from about 75% to about 85% by volume of the composition.

13. A pharmaceutical composition according to claim 11, wherein the composition contains a preservative.

14. A pharmaceutical composition according to claim 11, wherein the composition is preservative-free.

15. A pharmaceutical composition according to claim 11, wherein the composition contains an anesthetic agent.

16. (canceled)

17. A pharmaceutical composition according to claim 11, wherein the midazolam achieves a time to maximum plasma concentration (Tₘₐₓ) within about 5 minutes after intranasal administration.

18. A pharmaceutical composition according to claim 11, wherein the midazolam achieves a maximum plasma concentration (Cₘₐₓ) of about 40 ng/mL from a 2.5 mg dose or about 80 ng/mL from a 5 mg dose after intranasal administration.

19. (canceled)

20. A method of treating a mammal in need of rapid sedation, anxiolysis, amnesia, or induction of anesthesia comprising intranasally administering to the mammal a composition of claim 11, wherein the rapid sedation, anxiolysis, amnesia, or induction of anesthesia occurs within 5 minutes after intranasal administration.

21-26. (canceled)

27. The composition of claim 11, wherein the polyethylene glycol is polyethylene glycol 400.

28. The method of claim 20, wherein 5 mg of midazolam is administered to the mammal so as to provide rapid sedation, anxiolysis, or induction of anesthesia.

29. The composition of claim 11, wherein the composition comprises 25 mg/mL midazolam or a pharmaceutically acceptable salt thereof.
